(19) United States
(12) Plant Patent Application Publication
(10) Pub. No.: US 2003/0115648 P1
(43) Pub. Date: Jun. 19, 2003

(54) Nectarine tree named 'White Dragon'

(76) Inventor: Patrick Chiamori, Reedley, CA (US)

Correspondence Address: MARK D MILLER, KIMBLE, MACMICHAEL & UPTON, 5260 NORTH PALM AVENUE, SUITE 221, FRESNO, CA, 93704, US

(21) Appl. No.: 10/027,127

(22) Filed: Dec. 19, 2001

(51) Int. Cl. A01H 5/00

(52) U.S. Cl. PLT/188

(57) ABSTRACT

A new and distinct nectarine which is somewhat similar to the ‘Sunny Red’ nectarine tree (U.S. Plant Pat. No. 8534) from which it is a sport but from which it is distinguished by producing a later ripening fruit which are mature for harvesting and shipment approximately 3 to 5 days after the ‘Sunny Red’ nectarine, as well as being white fleshed as compared to the yellow fleshed fruit of ‘Sunny Red.’

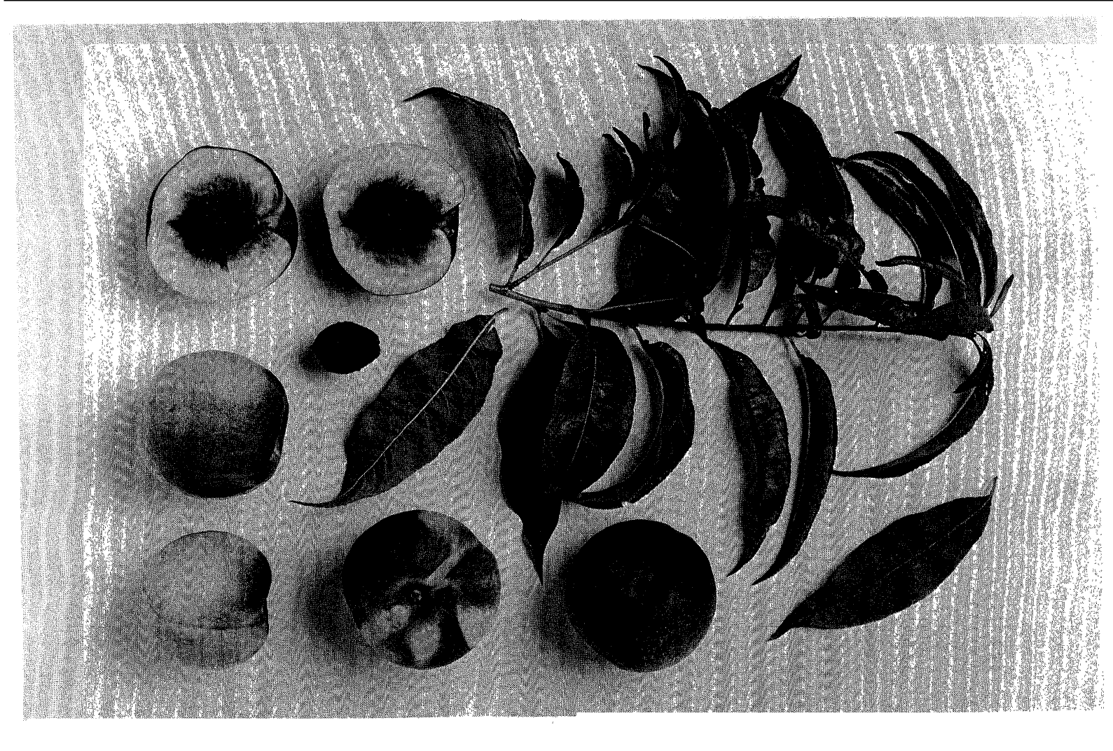

### BACKGROUND OF THE NEW VARIETY

The present invention refers to and relates to a new and distinct variety of nectarine tree which will hereinafter be denominated varietally as the ‘White Dragon’ nectarine tree which produces a clingstone fruit, which are mature for commercial harvesting and shipment approximately September 7-10 in the San Joaquin Valley of Central California as a late, fresh-market nectarine, white fleshed with a good red blush skin coloration.

In the development of new varieties of fruit trees a premium is placed upon those which produce fruit either early or late in the growing season when few competing varieties are available. When a new variety of fruit which matures early or late has good size, good color (blush), good flavor, and can be held or stored well, the fruit of this variety has a very good chance for commercial success in the fresh market. This new invention of a white fleshed nectarine meets this criteria.

### ORIGIN ABND SEXUAL REPRODUCTION OF THE NEW VARIETY

The present variety of nectarine tree was discovered by the inventor in his orchard of ‘Sunny Red’ nectarine trees (U.S. Plant Pat. No. 8534) which is located near Reedley, Calif. in the San Joaquin Valley of Central California. The inventor discovered the variety as a sport of a ‘Sunny Red’ nectarine tree In 1998. The new variety was first reproduced by the inventor in 1999 by hybrid grafting 12 trees from mature wood. The asexually reproduced trees first bore fruit of the new variety in September 2000. The inventor carefully compared the asexually reproduced trees with the parent sport including the fiuit thereof and has confirmed that the parent and the progeny are identical in all respects.

### SUMMARY OF THE NEW VARIETY

The ‘White Dragon’ nectarine tree is characterized by producing a clingstone fruit which has good red blush coloration and is ripe for commercial harvesting and shipment approximately September 7-10 in the San Joaquin Valley of Central California. The new variety is most closely similar to the ‘Sunny Red’ nectarine tree (U.S. Plant Pat. No. 8534) from which it is a sport but from which it is distinguishable in that it ripens about 3 to 5 days later than the fruit of the ‘Sunny Red’ nectarine tree and in that the fruit is somewhat larger in size than that of the ‘Sunny Red’ nectarine tree.

### BRIEF DESCRPTION OF THE DRAWING

The accompanying drawing is a color photograph showing several fruit of the new variety including the first in bottom view showing the apex end, the second in top view showing the stem cavity, the third in side view showing the surface, the fourth in side view to display the blush color, and the fifth to show a section fruit displaying the flesh and the pit cavity. The stone (or pit) is also displayed along with typical foliage of the new variety.

### DETAILED DESCRIPTION

Referring more specifically to the pomological details of this new and distinct variety of nectarine tree the following observations have been made for the ecological condition prevailing at the orchard of origin which is located near Reedley, Calif. in the San Joaquin Valley of Central California. All major color designations are by reference to the Dictionary of Color by Marez and Paul, First Edition, 1930. Common color names are also occasionally employed.

Tree: Generally develops and grows similar or equivalent to that of the parent ‘Sunny Red’ nectarine tree.

Vigor.—good.

Figure.—the tree is upright to upright spread.

Productivity.—productive.

Regularity of bearing.—regular under the ecological conditions of the Central San Joaquin Valley.

Trunk:

Size.—small to medium and the circumference taken approximately 10″ above ground is 7.079 inches. Tree is 3 years old.

Surface texture.—moderately rough.

Color code.—Log Cabin (15-A-5).

Lenticels.—Number — moderate Size — medium.

Branches:

Size.—small to medium; circumference of branches, approximately 20″ above ground is 3.927 inches. Scaffolds — 3 to 4 Angle of branches — 38°.

Surface texture.—a. Mature — relatively smooth b. Immature — smooth.

Color code.—One year or older — Tanagra Castilian Brown Old Cedar (9-J-10) Immature — Vindan Green (17-I-6).

Leaves:

Size.—medium to large.

Length.—122-136 mm. (4.80-5.35 inch).

Width.—40-44 mm. (1.57-1.73 inch).

Shape.—Lanceolate — leaf tip acuminate.

Color code.—a. Upwardly disposed surface, Chrome Green (23-H-12) b. Downwardly disposed surface, Parent Ar. (21-I-6).

Marginal form.—generally crenate.

Leaf vein.—a. Color Code — Callistre Green (119-L-6) b. Thickness — 2 mm (0.079 inch).

Glandular characteristics.—reniform.

Petiole.—Size — 2-2½ mm. (0.078-0.098 inch) Length — 10-14 mm. (0.39-0.55 inch) Thickness — 2-2½ mm. (0.079-0.098 inch).

Stem glands.—Stipules — small to medium, oblong to ovate — reddish brown, M&S Green (7-B-1).

Flowers:

Flower buds.—Length — medium — Average 10 mm (0.394 inch) Form — plump, somewhat conic shape Pubescence — pubescent Bud scales — very pubescent.

Flowers.—showy Size — medium diameter, average 34 mm (1.338 inch).

Pollen.—present, self fertile.

Petals.—5 L Length — Average 14 mm (0.551 inch) Width — Average 14 mm (0.551 inch) Margins — Slightly wavy.

Sepals.—5 broadly acute — Peony, Burmese Ruby+ (6-H-4).

Fragrance.—very slight.

Blooming period.—March 10 to March 17.

Color.—light pink (1-B-3).

Anthers.—average.

Stamens.—Length 10-12 mm (0.394-0.472 inch).

Filament.—light pink (1-H-4).

Pistil.—Average 18 mm (0.709 inch) Color — 1982 near Water Green.

Fruit:

Date of maturity — September 7-10.

Size.—Diameter Axial Plane — 66.68-82.55 mm. (2.85-3.25 inch) Transverse in Suture Plane — 66.68-75.61 mm. (2.625-2.94 inch) Transverse at Right Angles to Suture Plane — 61.9-69.85 mm (2.44-2.75 inch).

Form.—Uniformity — uniform Symmetrical/Asymmetric — symmetrical Suture — small with reddish orange, Cordial (5-A-5) Ventricle Surface — slightly uneven Stem Cavity: Width — 25 mm. (0.98 inch) Depth — 9-10 mm. (0.35 inch) Length — 35 mm. (1.37 inch) Shape — ovate Stem Short: Caliper — 4 mm. (0.16 inch) Apex — slightly rounded Pistil point — retuse

Skin.—Thickness — thin Texture — firm Tendency to Crack — not evident Color Code: a. Blush color — when textured, Romanesque (46-L-9 or 46-L-10) b. Ground color — Brass (11-L-6) Flesh Color: Popcorn (9-J-3) — there is a yellow area running from the epidermis along the suture line to the pit well, in the suture line itself (not illustrated in drawing because of cut along suture line) — Empire (9-K-3) Near the pit — reddish (42-L-1) Color of Pit Cavity — same color as flesh near pit Color of Pit Well — near Dahlid (43-L-6) Juice Production — very good Flavor — very good Aroma — moderate Texture — firm Fibers — see under stone.

Stone:

Free or cling.—Clingstone.

Fibers.—Numbers — many Length — 0.6-0.13 mm. (0.23-0.51 inch).

Size.—Length — 0.35-0.37 mm (1.38-1.46 inch) Width — 0.25-0.32 mm. (0.98-1.26 inch) Thickness — 0.20-0.30 mm. (0.79-1.18 inch).

Form.—ovate.

Apex shape.—with slightly pointed tip.

Color code (Dry).—Wineberg (55-H-4).

Base.—nearly flat.

Sides.—Slightly uneven, ventral edge relatively narrow, fourth groove produces a low reed on basal end.

Ridges.—grooves — particularly on apical end.

Tendency to split.—not known.

Use.—fresh market commercially.

Keeping quality.—very good.

Resistance to disease.—unknown.

Harvesting.—September 7-10.

Although the new variety of nectarine tree possesses the characteristics described above under the growing conditions prevailing near Reedley, Calif. in the Central San Joaquin Valley of California, it is to be understood that variations of the usual magnitude and characteristics as a result of change in growing conditions, irrigation, fertilization, pruning, pest control, climatic variation etc. are expected.

Having thus described and illustrated my new variety of nectarine tree, what I claim as new and desire to be secured by Plant Letters Patent is:

1. A new and distinct variety of nectarine tree substantially as illustrated and described which is somewhat nearly similar to the ‘Sunny Red’ (U.S. Plant Pat. No. 8534) from which it is a sport but from which it is distinguished by producing a later maturing fruit with good coloration, white fleshed with good edible quality, which matures for commercial harvesting and shipment September 7-10 or about 3-5 days after the fruit of ‘Sunny Red.’
